(19) United States
(12) Patent Application Publication
(10) Pub. No.: US 2007/0049980 A1
(43) Pub. Date: Mar. 1, 2007

(54) Trans-septal pressure sensor

(76) Inventors: Todd M. Zielinski, Minneapolis, MN (US); Douglas A. Hettrick, Blaine, MN (US)

Correspondence Address: MEDTRONIC, INC., 710 MEDTRONIC PARK, MINNEAPOLIS, MN, 55432-9924, US

(21) Appl. No.: 11/215,679

(22) Filed: Aug. 30, 2005

(51) Int. Cl. A61N 1/00

(52) U.S. Cl. 607/23

(57) ABSTRACT

A pressure sensor, in one embodiment, is passed through the atrial septal wall. Pivoting anchors secure the pressure sensor within the right atrium and flexible tines secure the pressure sensor from within the left atrium. Selectively pivoting the anchors permits adjustment of the radial span of the anchors, which may act as an electrode; thus, operable positioning of the electrode is adjustable.

### BACKGROUND OF THE INVENTION

1. Field of the Invention

The present invention relates to implantable medical devices. More specifically, the present invention relates to implantable medical devices that sense or measure pressure.

2. Description of the Related Art

There are a number of implantable medical devices (IMDs) that sense various physiological parameters and/or provide a variety of therapies. For example, implantable pulse generators (IPG) typically include one or more leads that are in contact with cardiac tissue to sense electrical depolarization and provide pacing stimuli. Implantable cardioverter/defibrillators (ICD) also typically include one or more leads and provide a larger stimulus for cardioversion or to defibrillate the heart. Often, IMDs include both pacing and cardioversion/defibrillation capabilities.

A housing containing the pulse generator, battery, capacitors, processor, memory, circuitry, etc. is implanted subcutaneously. One or more leads are delivered transvenously such that electrodes forming a portion of the lead are disposed within or contacting an outer portion of the heart. The housing, or “can”, may also include one or more electrodes that are selectively used in combination with the various lead electrodes.

In general, the leads sense electrical activity of the heart, typically represented as an electrogram (EGM), which is indicative of the cardiac depolarization waveform and indicates the timing of the various components of the complex. This data indicates whether and when intrinsic events occur, their duration and morphology. The timing of certain events (or their failure to occur when expected) is used to trigger various device actions. For example, sensing an atrial depolarization may begin a timer (an escape interval) that leads to a ventricular pacing pulse upon expiration. In this manner, the ventricular pacing pulse is coordinated with respect to the atrial event.

The heart includes four chambers; specifically a right and a left atrium and a right and left ventricle. Leads are commonly and routinely placed into the right atrium as well as the right ventricle. For left sided applications, the lead is typical guided through the coronary sinus and into a cardiac vein. One or more electrodes are then positioned (within the vein) to contact an outer wall of the left atrium and/or left ventricle. While direct access to the interior of the left atrium and left ventricle is possible, it has been historically less preferable. As the left ventricle provides oxygenated blood throughout the body, a foreign object disposed on the left side and providing a sufficient obstruction could lead to the formation of clots and would increase the risk that such a clot would form and be dispersed.

The sensing and utilization of electrical data is commonly employed as the electrodes used for delivering stimulus are typically also useful in sensing this data. This is generally non-problematic in left-sided applications as the electrical waveform is adequately sensed from the above described left side lead placement position.

A wide variety of other sensors are employed to sense parameters in and around the heart. For example, flow rates, oxygenation, temperature and pressure are examples of parameters that provide useful data in certain applications. Obtaining such data on the right side is typically non-problematic; however, obtaining the same data directly from the left side is made more difficult by the above noted desire to minimize invasiveness into the left atrium or ventricle.

Pressure data, in particular, is a useful parameter in determining the presence, status and progression of heart failure. Heart failure often leads to an enlargement of the heart, disproportionately affecting the left side. Left side pressure values would be useful in monitoring the patient's condition; gauging the effectiveness of a given therapy such as Cardiac Resynchronization Therapy (CRT); and timing, controlling or modifying various therapies.

Left atrial pressure, in particular, is a variable that defines the status of heart failure in a patient. Attempts have been made to measure surrogates of this variable by monitoring pulmonary wedge pressure in clinical care. Measurement of ePAD with implantable devices such as the Medtronic Chronicle™ have been used to measure real-time intracardiac chamber pressure in the right ventricle and provide an estimate of mean left sided pressure. These techniques generally do not provide certain phasic information and do not necessarily correlate with left atrial pressures under certain conditions such as pulmonary hypertension or intense levels of exercise.

### BRIEF DESCRIPTION OF THE DRAWINGS

FIG. 1 illustrates an implantable medical device (IMD) having a plurality of leads implanted within a heart.

FIG. 2 is a block diagram illustrating the functional components of an IMD.

FIG. 3 is an illustration of a heart showing an interior view of a right atrium and indicating the location of the fossa ovalis.

FIG. 4 is a schematic diagram illustrating a pressure sensor assembly in a deployed position.

FIG. 5 schematically illustrates an anchor member.

FIG. 6 is a schematic diagram of a plurality of anchor member and tine member.

FIG. 7 is a schematic diagram of a pressure sensor assembly positioned within a delivery catheter prior to implantation.

FIG. 8 is a schematic diagram of a pressure sensor assembly deployed from a delivery catheter.

FIGS. 9-11 illustrate an alternative embodiment wherein an anchor nut secures the deployed position of the anchors.

### DETAILED DESCRIPTION

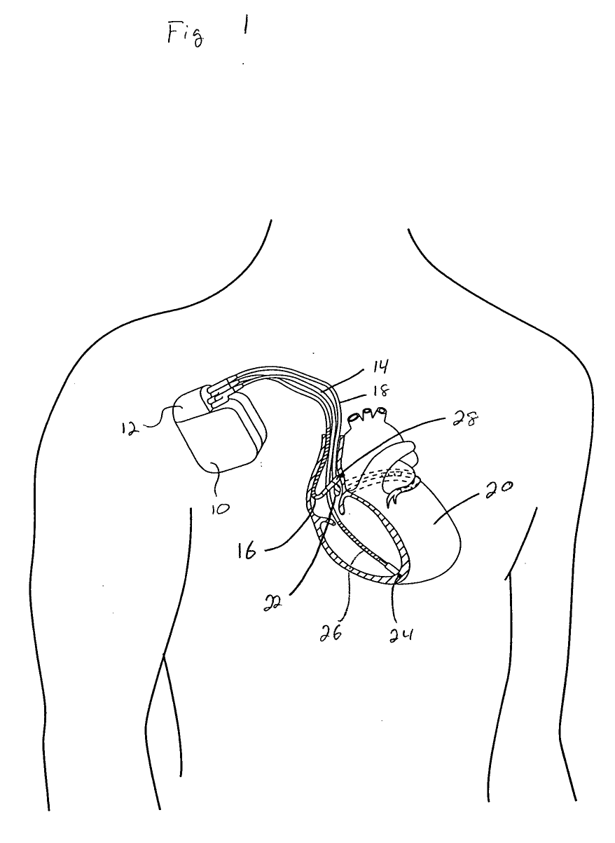

FIG. 1 illustrates an implantable medical device (IMD) 10 that includes pacing, cardioversion and defibrillation capabilities. A header block 12 forms a portion of the IMD 10 and three leads 14, 16, 18 are illustrated as coupled with the header block. A right ventricular lead 14 is disposed in the right ventricle of the heart 20. More specifically, a helical electrode tip 24 is embedded into the apex of the right ventricle. The electrode tip 24 forms or is part of a tip electrode and a coil electrode 26 is also included. A ring electrode may be disposed between the tip electrode 24 and the coil electrode 26.

An atrial lead 16 is disposed within the right atrium such than an electrode 28 contacts an interior wall of the right atrium. A left sided lead 18 is illustrated as passing through the coronary sinus 22 and into a cardiac vein. In this position, the left sided lead 18 has a distal end in contact with an outer wall of the left ventricle. The IMD 10 includes a housing that can act as an electrode or, though not illustrated, may include multiple electrodes. With such a configuration pacing stimuli is selectively delivered to the right atrium, the right ventricle, and/or the left ventricle. Likewise, a defibrillation pulse may be generated from any given electrode to any second electrode, such that the defibrillation waveform traverses the desired portion of the heart 20.

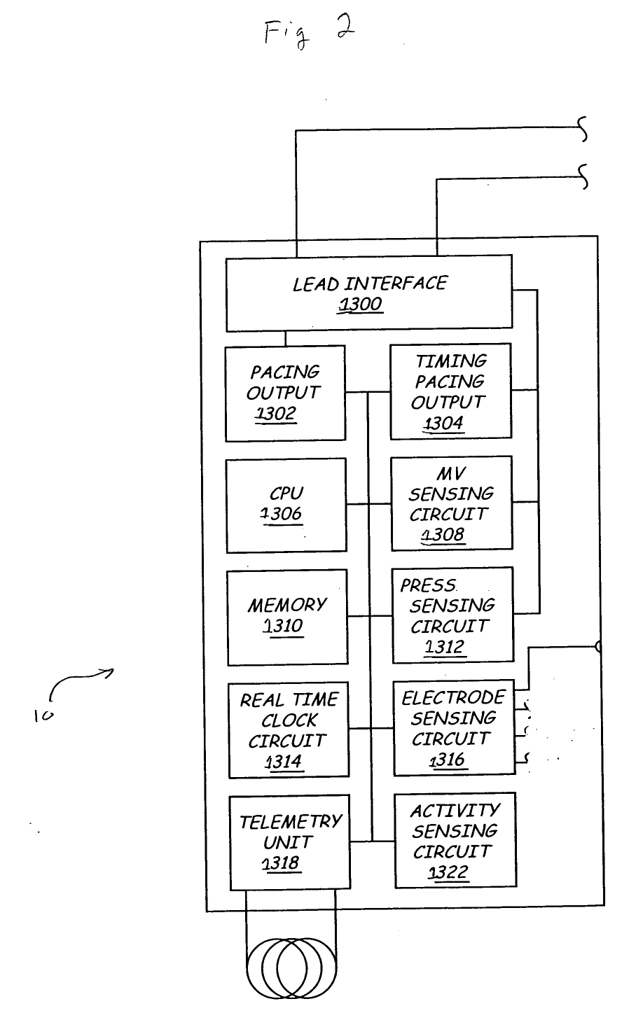

FIG. 2 is a simplified schematic diagram illustrating certain components of the IMD 10. The IMD 10 includes a processor or CPU 1306, memory 1310, timing circuits 1314, timing output circuit 1304, pacing and defibrillation output circuits 1302, an appropriate lead interface 300, and appropriate electrode sensing circuits 1316. The operation of the IMD 10 may be controlled by software or firmware and may be reprogrammed and/or provide data to an external device via telemetry unit 1318.

Also illustrated are exemplary sensing units that may be included with IMD 10. For example, an activity sensing circuit 1322, and a minute ventilation circuit 1308 are included. Thus far, IMD 10 is illustrated in an exemplary manner and may or may not include all components illustrated and may include many additional components and capabilities without departing from the spirit and scope of the present invention.

A pressure sensing circuit 1312 receives input from the pressure sensor described herein. In one embodiment, a pressure sensor is included on the right atrial lead 16 or a similar structure deployed within the right atrium. The pressure data, when received, is used by the CPU 1306 to monitor or control therapy, monitor the status of the heart, and/or to provide information to an external device via telemetry unit 1318. It should also be appreciated that various pressure sensors may provide relative data and an absolute pressure sensor (not shown) may be positioned external to the heart and utilized to provide reference data via telemetry unit 18 and/or to the external device.

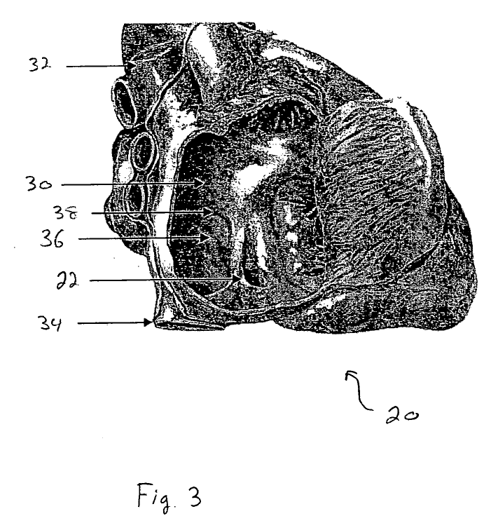

FIG. 3 is an illustration of the anatomy of a human heart 20. In particular, the interior of right atrium 30 is illustrated, along with the superior vena cava 32 and inferior vena cava 34. The atrial septum, dividing the right atrium from the left atrium is primarily defined (from the right side perspective, by the fossa ovalis 36. Surrounding the fossa ovalis 36 is the fossa limbus 38, which is a raised muscular rim. The fossa ovalis 36 is a relatively thin, but very strong membrane that separates the right atrium from the left atrium and is a non-conductive pathway for depolarization. The fossa ovalis 36 marks the previous location of the foramen ovale, which in embryonic and fetal development provided for direct passage between the atrial chambers. The fossa limbus 38 and the atrial tissue surrounding the fossa limbus 38 is conductive.

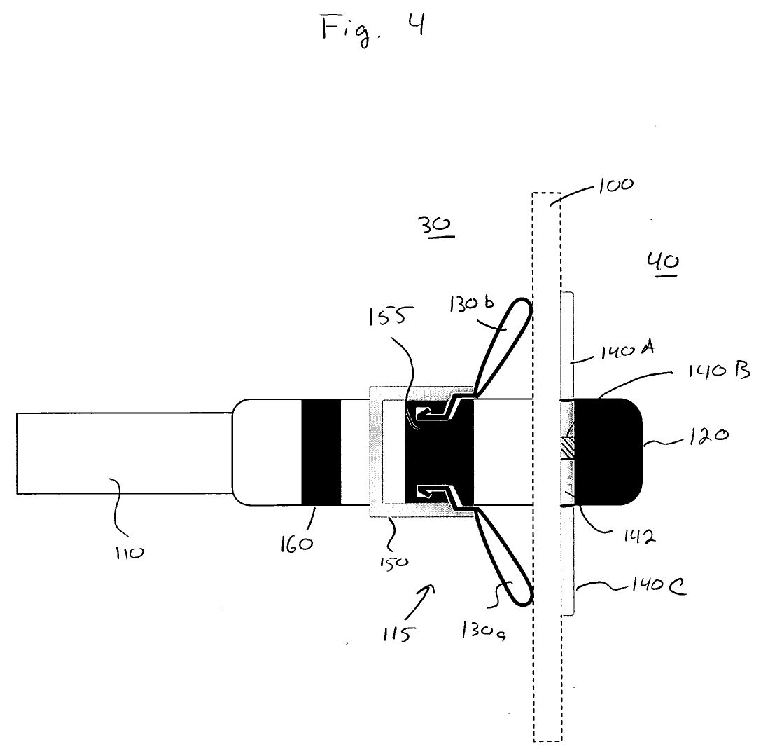

FIG. 4 is a schematic diagram illustrating a pressure sensor assembly 115 in a deployed position. A lead body 110 is deployed within the right atrium 30. The pressure sensor assembly 115 is operatively coupled with the distal end of the lead body 110. Typically, the proximal end of the lead body 110 will be coupled with the IMD 10 and though not illustrated, wires or other communication and/or therapy delivery mechanisms are disposed within the lead body 110.

A pressure sensor 120 is disposed on a distal portion of the pressure sensor assembly 115. In the deployed position, the pressure sensor assembly 115 passes through the septal wall 100 separating the right atrium 30 from the left atrium 40. Therefore, at least a portion of the pressure sensor 120 is positioned within the left atrium 40. In practice, the size of the protruding portion and the distance it protrudes are relatively small; thus, while permitting direct measurement of left atrial pressure there is no adverse effect on fluid flow leading to clotting. Furthermore, tissue growth about the protruding portion will further serve to minimize or even eliminate the amount of exposed surface area within the left atrium 40.

Intracardiac pressure sensing may be accomplished in a number of ways. The following US patents disclose a variety of pressure sensors and are herein incorporated by reference in their entireties: U.S. Pat. Nos. 6,223,081; 6,221,024; 6,171,252; 6,152,885; 5,919,221; 5,843,135; 5,368,040; 5,353,800; and 4,967,755. In the illustrated example, pressure sensor 120 includes a high fidelity pressure transducer mounted on a distal end of the pressure sensor assembly 115 and is configured for placement within the left atrium. The present invention may also be employed to deliver a pressure sensor 120 into the left ventricle through the ventricular septal wall from the right ventricle. Mechanically, the present invention will operate in the same manner as described herein with appropriate dimensional changes. The ventricular septal wall is thicker than the atrial septal wall 100 and makes passage therethrough more difficult. The process is further complicated by the location of the Bundle of His, which if intact is preferably avoided during the implantation process. The present invention would also provide a mechanism for His bundle pacing. Thus, while the embodiments are described with respect to atrial placement, the invention is not so limited and includes placement and use within the ventricles.

Phasic information of the left atrial pressure provided by the pressure sensor 120 can be used, for example, by the IMD 10 to control several pacing parameters such as AV timing and VV timing for management of AF and CHF by optimizing left sided filling and ejection cycles and enhance cardiovascular hemodynamic performance. Such data may also be used for assessment of mitral regurgitation and stenosis. For device based management of atrial fibrillation, the phasic information can be used for discriminating atrial fibrillation from flutter and optimizing atrial anti-tachycardia pacing therapies.

Pressure sensor 120 provides diagnostic data to clinicians and/or control device operation by automated feedback control. Direct, real-time left atrial pressure measurement may be utilized to provide diagnostic information for management of heart failure and in patients with pacemakers, to optimize pacing parameters to prevent its progression. In addition, pressure sensor 120 provides information about the atrial substrate for management of AF and may control pacing parameters to prevent progression of AF. Reference is made to U.S. patent application Ser. No. 11/097,408, filed on Mar. 31, 2005 and titled “System and Method for Controlling Implantable Medical Device Parameters in Response to Atrial Pressure Attributes,” which is herein incorporated by reference in its entirety.

As indicated, the pressure sensor assembly 115 passes through an opening in the septal wall 100. This may occur at the fossa ovalis 36, where the septal wall is relatively thin; though this location is not mandated for the present invention. Once positioned, the pressure sensor assembly 115 is held in place by anchors 130 disposed on one side of the septal wall 100 acting in opposition to tines 140 acting on the other side of the septal wall 100. In other words, the anchors 130 and tines 140 “sandwich” the septal wall 100 between them. While various embodiments are illustrated representing anchor and tine combinations, it should be readily apparent that numerous variations exist that are within the scope of the present invention.

With continued reference to FIG. 4, anchors 130A and 130B are illustrated. Anchors 130A and 130B engage and are retained within anchor base 155. As will be described in greater detail, the anchors 130 may flex or pivot towards or away from the septal wall 100 while being retained within the anchor base 155. Anchor sleeve 150 acts as a retaining member to prevent the anchors from pivoting away from the septal wall 100 (as illustrated) after being properly positioned.

As described thus far, the pressure sensor assembly 115 is retained in position so that pressure sensor 120 is disposed within the left atrium 40 and is capable of providing mean or dynamic real time or near real time pressure values. These values may be relative or absolute when correletated to an external (to the heart) pressure reference sensor (not shown). In addition to pressure data, the pressure sensor assembly 115 may include one or more electrodes to deliver electrical stimulation and/or sense electrical activity. A proximal electrode 160 is illustrated in a proximal portion of the pressure sensor assembly 115 and is not in contact with tissue. As such, it may function in a manner similar to a ring electrode. The proximal electrode 160 may be used, for example, to provide EGM data. The anchors 130 may be electrically conductive or include portions that are electrically conductive such that the anchors 130 function as either a single collective electrode or individual independent electrodes. As illustrated, the anchor base 155 provides a common electrical point to which the various anchors 130 are attached. As the anchors 130 are in contact with tissue, they may be used to provide electrical pacing stimuli and of course, sense electrical activity. The location selected for placement of the pressure sensor 120 determines the proximity of the assembly 115 to conductive tissue. For example, as noted above, the fossa ovalis 36 is typically non-conductive while the surrounding fossa limbus 38 is conductive. Thus, the anchors 130 can be selected and adjusted to not only retain the assembly 115 in the proper position, but also to contact conductive tissue to act as a pacing electrode.

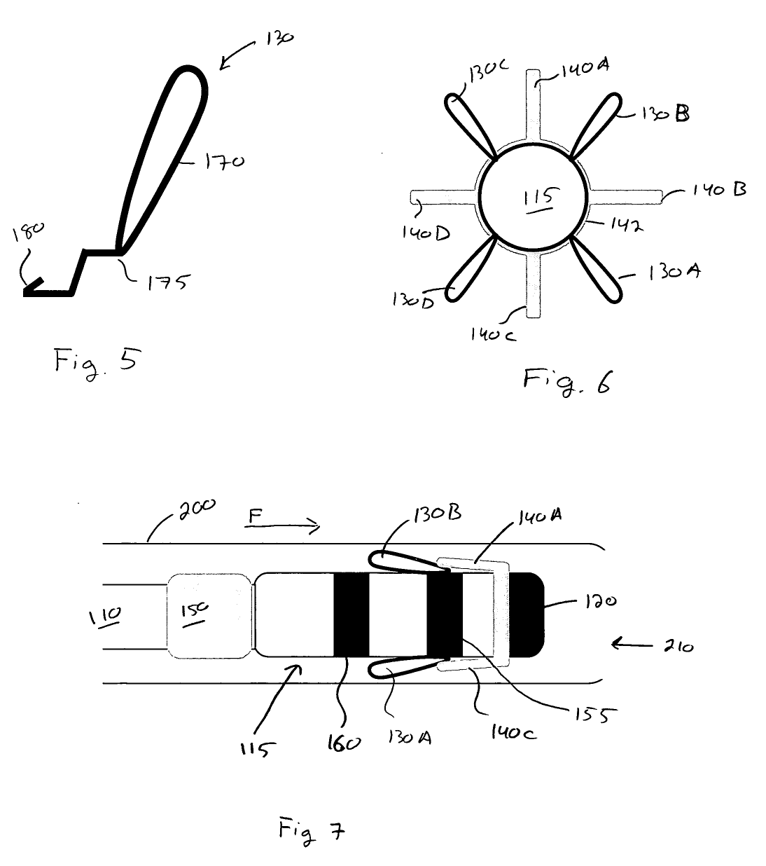

FIG. 5 schematically illustrates one anchor 130. The anchor 130 includes a contact arm 170 coupled via a flex point 175 to a locking tip 180. The locking tip 180 is inserted into the anchor base 155 and retained. The contact arm 170 is able to pivot freely or with little resistance about the flex point 175. The contact arm 170 has a strength, size and shape sufficient to abut cardiac tissue and retain the assembly in the selected position. As indicated, the control arm 170 may be made from a biocompatible electrically conductive material, a portion may be electrically conductive, or the entire arm may be non-conductive, thus providing only an anchoring function.

FIG. 6 is a schematic end view illustrating anchor base 115 with four anchors 130A-130D attached thereto. In addition, a portion of a tine support 142 coupled with a distal end of the anchor base 115 is visible along with four tines 140A-140D depending therefrom. The relative angular positioning illustrated between the anchors 130 and the tines 140 permits all elements to be viewed; however, there is no requirement to provide or maintain such an alignment. Furthermore, more or fewer tines 140 and/or anchors 130 may be utilized in any given embodiment.

The tines 140 and tine support 142 are made from an appropriate biocompatible material that permits the tines 140 to fold towards the assembly 115 during implantation to extend to the position illustrated in FIG. 6 after piercing the septal wall 100. In one embodiment, the tines 140 and support 142 are made of silicone. The natural resiliency of the silicone causes the tines 140 to extend when permitted.

Alternatively, various metals or other materials may be employed that utilize resilient characteristics, shape memory, activated shape memory (e.g., heat activated), or are mechanically deployed. Such deployment may occur by retracting (or effectively retracting by deployment of the anchors 130) the assembly 115 after piercing the septal wall 100. The wall 100 will contact the tines 140 and cause them to deploy. Alternatively, the tines may be mechanically deployed from within the lead body 110 by, for example, guide wires, a stylet or the like. In the illustrated embodiment, the tines 140 are silicone and minimally obtrusive. Thus, their presence will have little impact on the left atrium and will likely lead to tissue encapsulation.

It should be appreciated that the tines 140 in alternative embodiments could include conductive material or pathways and serve as pacing and/or sensing electrodes. As such, members similar to or identical to anchors 130 could be utilized as tines rather than the illustrated tines 140. For purposes of the present disclosure, the term anchor refers to the mechanisms on the lead side of the intended anatomical structure while the term tine refers to the mechanism intended to be deployed on a side of the anatomical structure opposite the lead body.

FIG. 7 is a schematic diagram of a pressure sensor assembly 115 positioned within a delivery catheter 200 prior to implantation. The sleeve 150 is positioned proximally with respect to the anchors 130. Thus, the anchors 130 fold, flex or pivot towards the assembly 115 so that they are accommodated within the diameter of the delivery catheter 200. Similarly, the tines 140 are flexed in a similar manner.

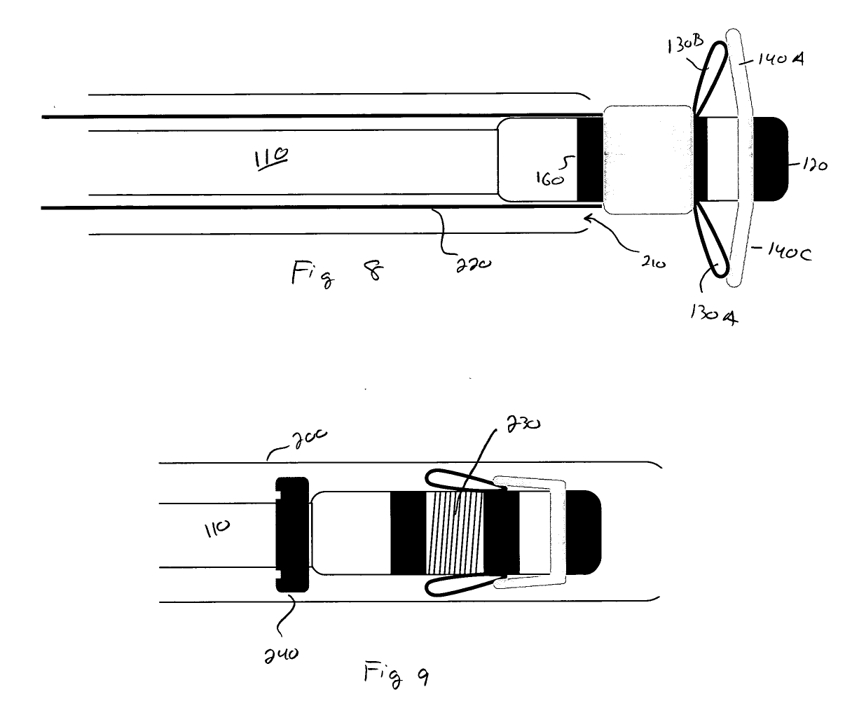

FIG. 8 schematically illustrates a sleeve deployment tool 220 in contact with the sleeve 150. The assembly 115 has been advanced beyond the distal end of the delivery catheter 200 through distal opening 210. The assembly 115 may be advanced to this position in any number of ways including using a stylet, simply advancing the lead body 100, or by using sleeve deployment tool 220 which essentially acts as an external stylet. The sleeve deployment tool 220 is a tool operable from a proximal portion of the lead 110 that allows sufficient force to be exerted against sleeve 150 to cause sleeve 150 to advance.

For implantation, the delivery catheter 200 is delivered to the target site and the distal opening is placed against the right atrial septal wall 100 where the pressure sensor 120 will pierce into the left atrium. Through one of the above described mechanisms, the assembly 115 is advanced distal to the delivery catheter 200 and the pressure sensor 120 (or a portion thereof) and the tines 140 pass into the left atrium. Though not illustrated, the pressure sensor assembly 115 may include a piercing tip to facilitate puncturing the septal wall. Alternatively, a piercing device, e.g., an appropriate gauge needle, may be delivered via the delivery catheter 200 and caused to puncture the septal wall 100. The piercing device is retracted and the assembly 115 is delivered.

When properly positioned, the proximal end of the lead body 110 is retained and the sleeve deployment tool 220 is advanced. This causes the sleeve 150 to move distally and to pivot the anchors 130 towards the septal wall (as illustrated in FIG. 8). The sleeve 150 is constructed so that once positioned, it retains the anchors 130 in the deployed position. In one embodiment, the sleeve 150 is a silicone ring that frictionally engages the pressure sensor assembly 115 so that its positioned is retained. FIG. 8 illustrates a deployed device absent the septal wall 100. As such, the anchors 130 contact the tines 140. When actually implanted, as illustrated in FIG. 4, the tines 140 are in contact with the septal wall 100 in the left atrium and the anchors 130 contact the septal wall 100 within the right atrium; more specifically, the electrically

FIGS. 9-11 illustrate an alternative embodiment wherein an anchor nut 240 is used instead of the sleeve 150. In this embodiment, an anchor nut deployment tool, controlled from a proximal end of delivery catheter 200 is utilized to rotate the anchor nut 240. The assembly 115 includes a threaded anchor base 230. The anchor nut 240 is slid distally until reaching the threaded anchor base 230, then rotated to cause further distal movement. The anchor nut 240 deploys the anchors 230 by pivoting them towards the septal wall (or distal from the catheter 200).

It should be appreciated that by using either the anchor nut 240 or the anchor sleeve 150, the amount or degree to which the anchors 130 are pivoted is controllable. Thus, the tension imparted against the septal wall 100 is adjustable. Furthermore, the radial distance from the center of the puncture (e.g., center of pressure sensor 120) is also variable. That is, the anchor 130 will achieve its greatest radial distance when positioned orthogonally to the main axis of the sensor assembly 115. The more acute the angle between the anchor 130 and the when the anchor 130 is used as an electrode and needs to contact conducting cardiac tissue. If the sensor assembly 115 is deployed through conductive tissue, then there is likely no issue. Alternatively, if the sensor assembly 115 is deployed through non-conductive tissue (e.g., the fossa ovalis 36), then the anchors 130 need to extend to conducting tissue (e.g., the fossa limbus 38) to act as a pacing electrode.

Where this is a concern, the angle is adjusted to give an appropriate amount of tension as well as provide an appropriate radial distance so contact is made with conductive tissue. If this is insufficient, the puncture site may be selected (e.g., off center) so that at least one anchor 130 is capable of reaching conductive tissue. In addition, multiple sensor assemblies 115 may be provided that include anchors 130 having various lengths so that an appropriate assembly 115 is selected based upon a given patient's actual anatomy.

Regardless of whether the puncture site is through conductive tissue, threshold testing is utilized to determine if the anchors 130 are properly positioned to act as pacing/sensing electrodes. If not, the position of the anchors 130 is adjusted until the threshold testing provides satisfactory results.

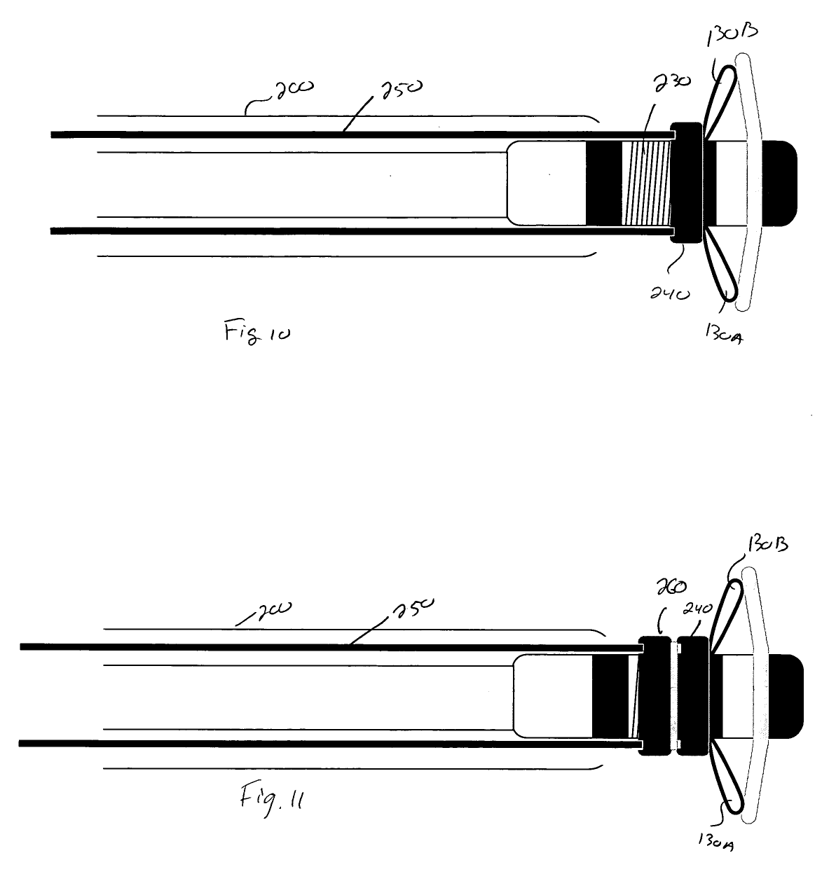

Returning to FIG. 10, the anchor nut 240 is rotated via the anchor nut deployment tool 250 until advanced to the desired position. In one embodiment, this completes the implantation procedure. FIG. 11 illustrates an embodiment including a locking nut 260. After the anchor nut 240 is deployed, the deployment tool 250 is retracted. The locking nut 260 is advanced over the lead 110 until reaching the threaded base 230. Then, using the deployment tool 250, the locking nut 260 is rotated until it firmly abuts the anchor nut 240. This prevents the anchor nut 240 from inadvertent reversal and movement of the anchors 130.

As disclosed herein, a number of embodiments have been shown and described. These embodiments are not meant to be limiting and many variations are contemplated within the spirit and scope of the invention, as defined by the claim. Furthermore, particular elements illustrated and described with respect to a given embodiment are not limited to that embodiment and may be used in combination with or substituted into other embodiments.

We claim:

1. An implantable medical device (IMD) comprising:
a lead body;
an assembly housing disposed at a distal end of the lead body;
a pressure sensor coupled with the assembly housing;
an anchor coupled to the assembly housing;
an anchor retention member that engages the assembly housing and selectively bias the anchor in a first direction such that the anchor biases the assembly housing in a second direction; and
a tine coupled to the assembly housing, the tine moveable between a first position and a second position, wherein the opposes movement of the assembly housing in the second direction.

2. The IMD of claim 1, further comprising a first electrode disposed along the lead body proximal from the anchor.

3. The IMD of claim 2, wherein the first electrode is coupled with the assembly housing.

4. The IMD of claim 1, wherein the anchor is pivotably coupled to the assembly housing.

5. The IMD of claim 4, wherein advancement of the anchor retention member in a distal direction towards the pressure sensor causes the anchor to pivot.

6. The IMD of claim 5, wherein a radial distance from the assembly housing to a distal tip of the anchor is variable based upon the position of the anchor retention member.

7. The IMD of claim 6, wherein the anchor is an electrode.

8. The IMD of claim 1, wherein the anchor is an electrode.

9. The IMD of claim 1, wherein the anchor retention member is a sleeve that slidably engages the assembly housing.

10. The IMD of claim 9, wherein the sleeve is silicone.

11. The IMD of claim 1, further comprising a threaded track disposed on the assembly housing, wherein the retention member is an anchor nut that selectively enrages the threaded track such that rotation of the anchor nut causes travel along the assembly housing.

12. The IMD of claim 11, further comprising a locking nut that engages the threaded track proximal to the anchor nut.

13. The IMD of claim 1, wherein the tine includes a silicone ring coupled with the assembly housing and further including a plurality of depending silicone tine substrates extending radially from the ring.

14. An implantable medical device comprising:
a lead body;
means for sensing pressure coupled with a distal end of the lead body; and
means for securing the means for sensing pressure to a substrate through which the means for sensing pressure passes.

15. The IMD of claim 14, wherein the means for securing include means for anchoring that are adjustable to vary a radial distance between a distal end of the means for anchoring and a housing to which the anchoring means are coupled.

16. The IMD of claim 15, wherein the means for anchoring further act as an electrode.

17. The IMD of claim 14, wherein the means for securing further include a plurality of tines deployable from a retracted position to an extend position wherein the tines extend in a radial direction.

18. The IMD of claim 14, wherein the means for securing includes an advanceable sleeve engageable with a pivotable anchor member.

19. The IMD of claim 14, wherein the means for securing includes an anchor nut wherein rotation of the anchor nut causes linear travel so that the anchor nut engages a pivotable anchor member.

20. The IMD of claim 19, wherein the means for securing further include means for locking the anchor nut in a given position.
